(19) United States
(12) Patent Application Publication
(10) Pub. No.: US 2016/0135702 A1
(43) Pub. Date: May 19, 2016

(54) Techniques for Predicting Cardiac Arrhythmias Based on Signals from Leads of Electrocardiography

(71) Applicant: THE BOARD OF TRUSTEES OF THE LELAND STANFORD JUNIOR UNIVERSITY, Palo Alto, CA (US)

(72) Inventor: Marco V. Perez, Palo Alto, CA (US)

(73) Assignee: The Board of Trustees of the Leland Stanford Junior University, Palo Alto, CA (US)

(21) Appl. No.: 14/899,901

(22) PCT Filed: Jun. 20, 2014
(86) PCT No.: PCT/US14/43338

§ 371 Date: Dec. 18, 2015

Related U.S. Application Data
(60) Provisional application No. 61/837,919, filed on Jun. 21, 2013.

(51) Int. Cl. A61B 5/046; A61B 5/00; A61B 5/044; A61B 5/0408; A61B 5/04

(52) U.S. Cl. CPC A61B 5/046; A61B 5/0408; A61B 5/04012; A61B 5/044; A61B 5/4836

(57) ABSTRACT

Techniques for predicting cardiac arrhythmia includes obtaining first data that indicates an electrocardiography recording from a patient; and, automatically deriving, on a processor, P-wave characteristics on a plurality of leads of the electrocardiography recording. A value for a first parameter, Pindex3, is determined based on a standard deviation of P-wave duraations automatically derived from only three leads of the plurality of leads. A risk of incidence of cardiac arrhythmia for the patient is determined based, at least in part, on the first parameter, Pindex3.

### BACKGROUND OF THE INVENTION

Atrial fibrillation (AF) is a cardiac arrhythmia characterized by disorganized atrial electrical activity leading to loss of effective contraction. A trial fibrillation affects more than 2.2 million people in the United States, accounts for approximately 75,000 strokes per year, and is independently associated with a 1.5-fold to 1.9-fold increase risk of death. It is associated with increased morbidity, ICU length of stay, and total length of stay.

The primary goals of therapy for AF involve minimizing symptoms caused by AF through rhythm or rate control and lowering stroke risk with anticoagulant therapy. However, roughly 6.5% of patients who present with AF-related strokes have no prior known history of AF. The ability to predict onset of AF may identify a group of patients whose stroke risk can be modified. For example, prediction is especially useful in post-operative cardiac patients who are subject to the development of AF. Effective chemical prophylaxis exists for AF. However, significant adverse events, such as bradycardia and heart block, are associated with prophylactic therapy. Thus, the benefits of indiscriminate use of prophylactic therapy have not been shown to outweigh the risks. However, confining prophylactic therapy to patients at high risk of developing postoperative atrial fibrillation (POAF), for example, can improve the benefit/risk profile. Prediction models based on clinical variables have not been sufficiently robust to guide POAF prophylactic therapy.

The 12-lead resting electrocardiogram (ECG) is most frequently used in the evaluation of patients for cardiovascular disease and, because of its relatively low cost, has the greatest potential to be used as a screening tool. Maximal P-wave duration (Pmax) and P-wave dispersion (Pdisp), defined as the difference between the Pmax and Pmin across the 12 ECG leads, have been consistently reported as predictors of postoperative AF, or frequent episodes of AF paroxysms, or recurrence of AF after cardioversion. Most of these studies were performed on a select group of patients known either to have an established history of AF or to be at high postoperative risk of AF. One recent population-based study identified Pmax and P-wave morphologies as very strong predictors of AF. However, these studies relied on manual, and often meticulous, measurements of the P waves.

### SUMMARY OF THE INVENTION

Techniques are provided for predicting incidence of cardiac arrhythmias. The primary arrhythmia of interest is atrial fibrillation, though one or more of the same parameters are anticipated to predict other arrhythmias such as atrial flutter.

In a first set of embodiments, a method includes obtaining first data that indicates an electrocardiography recording from a patient. The method includes automatically deriving, on a processor, P-wave characteristics on a plurality of leads of the electrocardiography recording. The method also includes determining a value for a first parameter, Pindex3, based on a standard deviation of P-wave durations automatically derived from only three leads of the plurality of leads. The method still further includes determining a risk of incidence of cardiac arrhythmia for the patient based, at least in part, on the value of Pindex3.

In one or more other sets of embodiments, a computer-readable medium, apparatus or system is configured to cause one or more steps of the above method to be performed.

Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The present invention is illustrated by way of example, and not by way of limitation, in the figures of the accompanying drawings, in which like reference numerals refer to similar elements and in which:

FIG. 1A and FIG. 1B are block diagrams that illustrate example placement of electrodes and derivation of lead signals for electrocardiography in the prior art;

FIG. 1C is block diagram that illustrates example time series signals from multiple leads of an electrocardiogram;

FIG. 1D is block diagram that illustrates example components of a heartbeat signal in a single lead of an electrocardiogram;

FIG. 2 is a block diagram that illustrates an example system to predict the incidence of a cardiac arrhythmia, according to an embodiment.

FIG. 3 is a flow diagram that illustrates an example method to predict the incidence of a cardiac arrhythmia, according to an embodiment;

FIG. 4 is a block diagram that illustrates a computer system upon which an embodiment of the invention may be implemented; and

FIG. 5 illustrates a chip set upon which an embodiment of the invention may be implemented.

### DETAILED DESCRIPTION

Techniques are described for predicting cardiac arrhythmia as a risk of incidence of such arrhythmia based on signals from three or more leads of electrocardiography. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Some embodiments of the invention are described below in the context of determining the risk of incidence of atrial fibrillation as a hazard ratio. However, the invention is not limited to this context. In other embodiments the same parameters are used to determine the risk of incidence of the same or other arrhythmias, such as atrial flutter and atrial tachycardia, using the hazard ratio or other statistic, such as correlation coefficient.

### 1. Overview

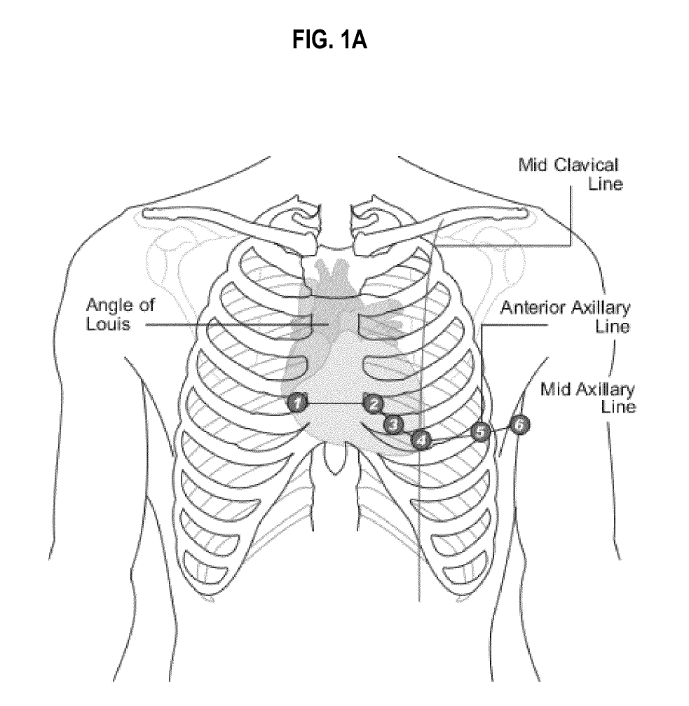

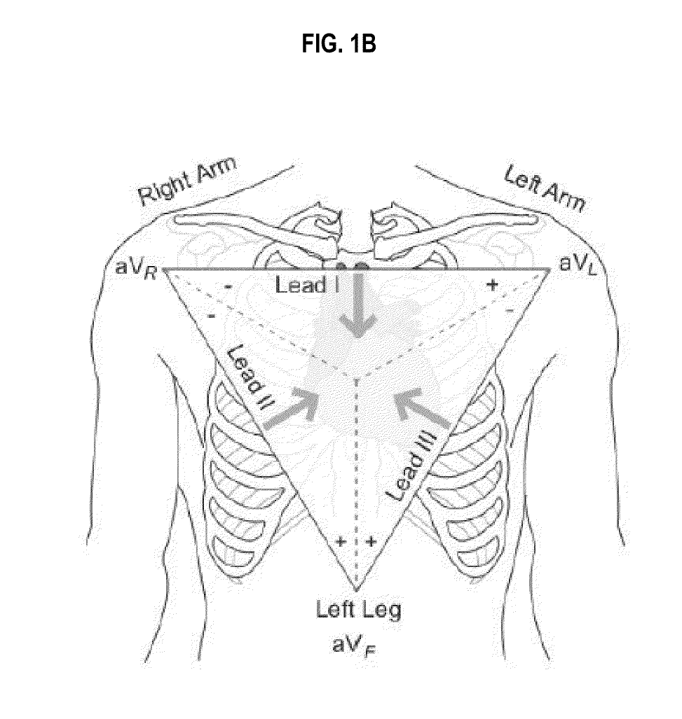

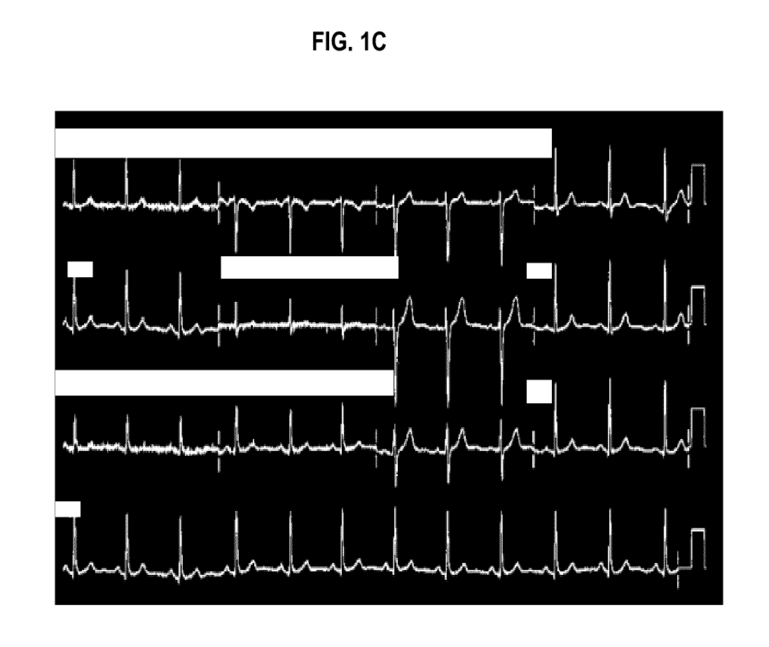

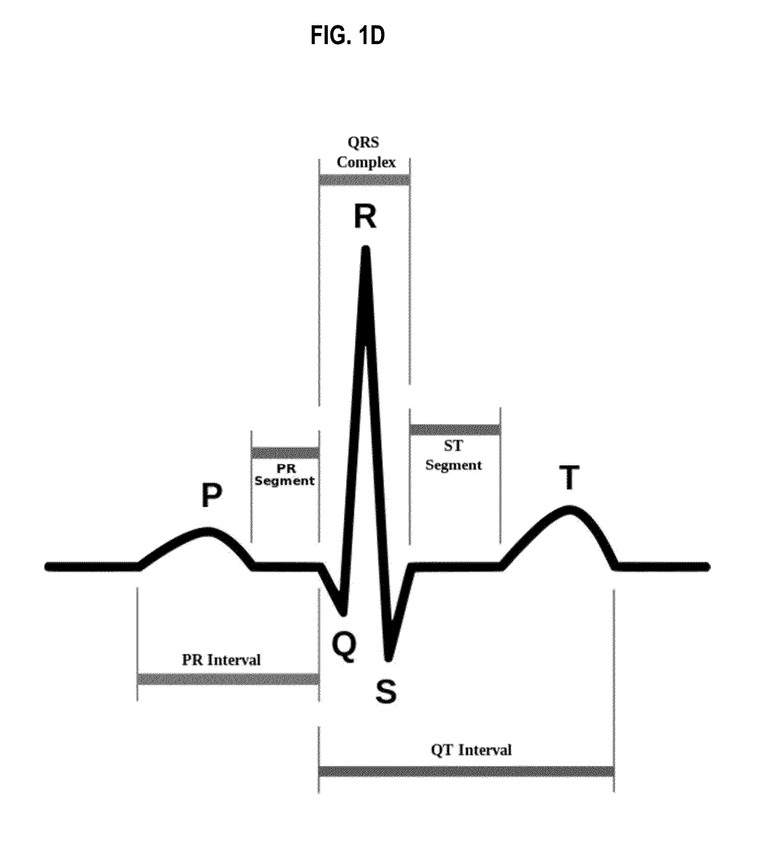

FIG. 1A through FIG. 1B are block diagrams that illustrate example placement of electrodes and derivation of lead signals for electrocardiography in the prior art. For the common 12 lead electrocardiography, electrodes are placed at six thorax positions marked as numbered red circles in FIG. 1A. The electrodes at these positions produce data channels, called leads, which are referenced as V1 through V6, respectively. In addition to the six electrodes that provide leads V1 through V6, three other electrodes are used as references, one on the right arm or shoulder, one at the left arm or shoulder, and one at the foot or hip. These electrodes produce data channels (again called leads) referenced as aV_{R}, aV_{L} and aV_{F}, respectively, as depicted in FIG. 1B. Three other data channels (again called leads), and referenced as lead I, lead II and lead III, respectively, are derived from leads aV_{R}, aV_{L} and aV_{F}. As depicted in FIG. 1B, lead I is derived by subtracting lead aV_{R} from aV_{L}; lead II is derived by subtracting lead aV_{R} from aV_{F}; and, lead III is derived by subtracting lead aV_{L} from aV_{F}. The 9 electrode-based and 3 derived leads provide the 12 leads for 12 lead electrocardiography. A recorded time series from all of the 12 leads is called a 12 lead electrocardiogram. Time series of the electrocardiogram are on the order of about ten seconds to about one minute.

FIG. 1C is block diagram that illustrates example time series signals from multiple leads of an electrocardiogram from a normal patient. These time series are on the order of ten seconds and show the time series from leads I, II, III and V1, respectively. About a dozen heartbeats are recorded on each lead in the ten seconds. Each heartbeat is characterized by various signal components resulting from electrical pulses that pass through various tissues and chambers of the heart.

FIG. 1D is a block diagram that illustrates example signal components of a heartbeat signal in a single lead of an electrocardiogram. Vertical distance indicates amplitude of electrical signal and horizontal distance indicates differences in time. This is an idealization of the signal components of a single heartbeat and both the amplitude and time scales are arbitrary. The characteristics of an individual heartbeat include a P-wave labeled P in FIG. 1D, followed by a QRS complex so labeled in FIG. 1D, followed by a T-wave labeled T in FIG. 1D. The presence, absence, amplitude, duration and temporal separation of these components and statistics based on multiple observations of each on one or more leads constitute the characteristics of the heartbeat. Some characteristics are depicted in FIG. 1D, including: a PR interval encompassing the P-wave and preceding the QRS complex; a PR segment following the P-wave and preceding the QRS complex; amplitude dips at points Q and S bracketing a peak at point R of the QRS complex; ST segment following the QRS complex and preceding the T-wave; and a QT interval encompassing the QRS complex and the T-wave.

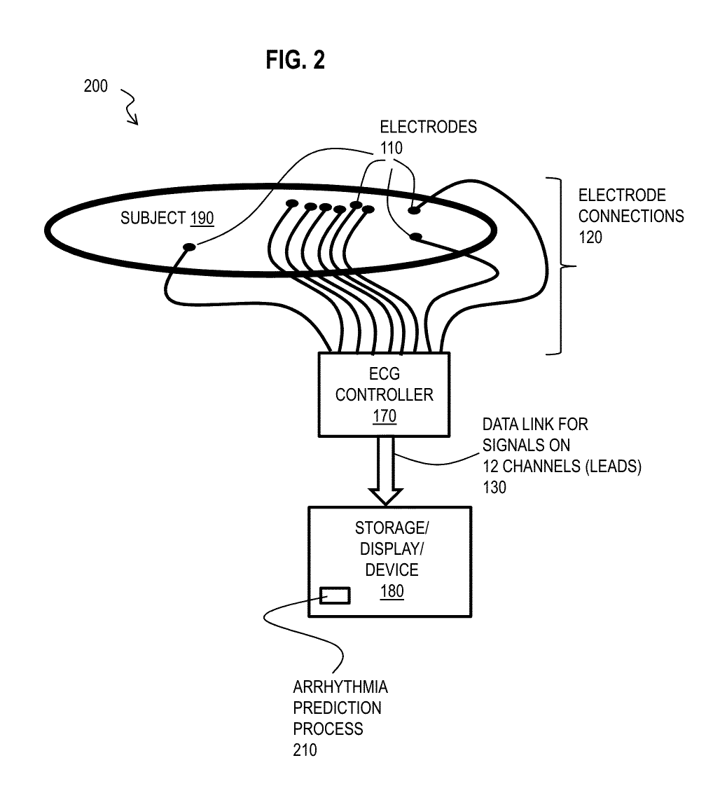

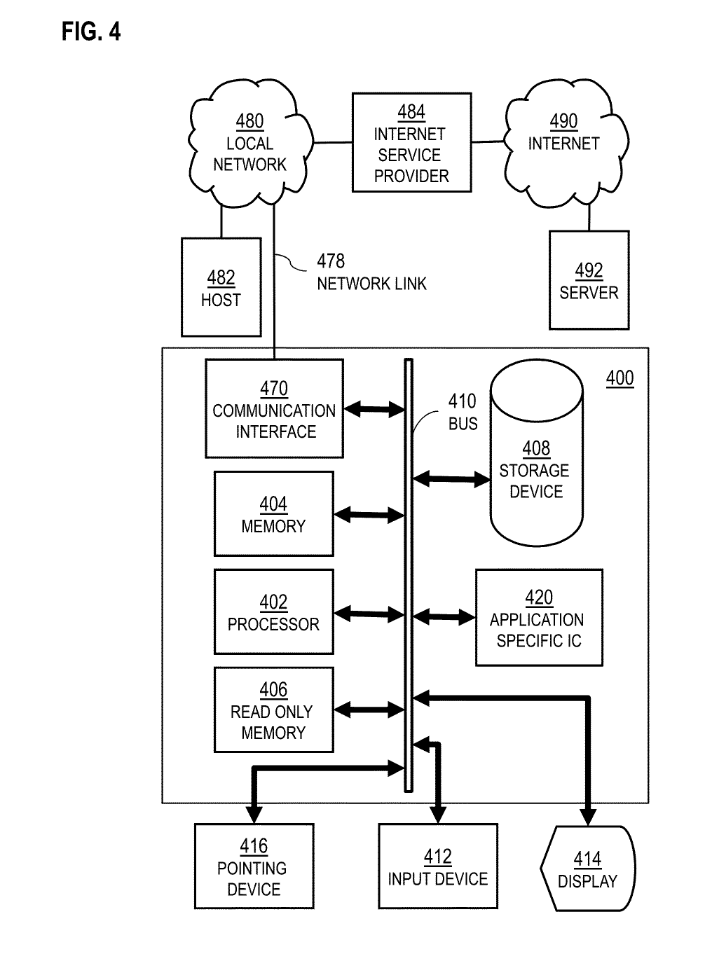

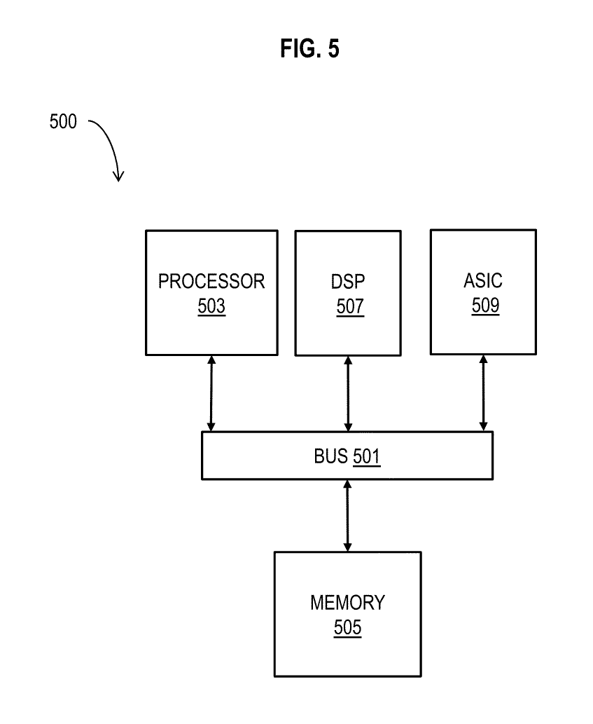

FIG. 2 is a block diagram that illustrates an example system to predict the incidence of a cardiac arrhythmia, according to an embodiment. Although a subject 190, such as a patient, is depicted in FIG. 2 for purposes of illustration, the subject 190 is not part of the system 200. The system 200 includes electrodes, such as the 9 electrodes described above with reference to FIG. 1A and FIG. 1B, and connections 120 connecting the electrodes 110 to a electrocardiograph controller 170. The controller 170, determines the derived leads and outputs signals for the various leads, such as the 12 leads of 12 lead electrocardiography, on a data link 130. The data passed on data link 130 are stored or displayed or both on a device 180. In some embodiments the device 180 includes a processor, such as a computer described in more detail below with reference to FIG. 4, or a chip set described in more detail below with reference to FIG. 5.

In some embodiments, the data link 130 is a direct wired connection; but, in other embodiments, the data link is a wireless connection or a wired or wireless network connection, as described below with reference to FIG. 4. In some embodiments, analog data is transmitted over data link 130 and stored on analog storage or displayed on an analog display, or both. In some embodiments, device 180 includes an analog to digital converter (ADC) to convert analog data to digital data. In some embodiments, controller 170 includes an ADC and outputs digital data over data link 130.

In various embodiments, the system 200 includes an arrhythmia prediction process 210 executing on a processor. As depicted, the process 210 executes on a processor of device 180. In other embodiments, data is transferred to a remote processor and process 210 runs, in whole or in part, on one or more remote processors.

Although processes, equipment, and data structures are depicted in FIG. 2 as integral blocks in a particular arrangement for purposes of illustration, in other embodiments one or more components or processes or data structures, or portions thereof, are arranged in a different manner, on the same or different hosts, in one or more databases, or are omitted, or one or more different processes or data structures are included on the same or different hosts. For example, in some embodiments, process 210 executes on one or more processors including one or more remote processors.

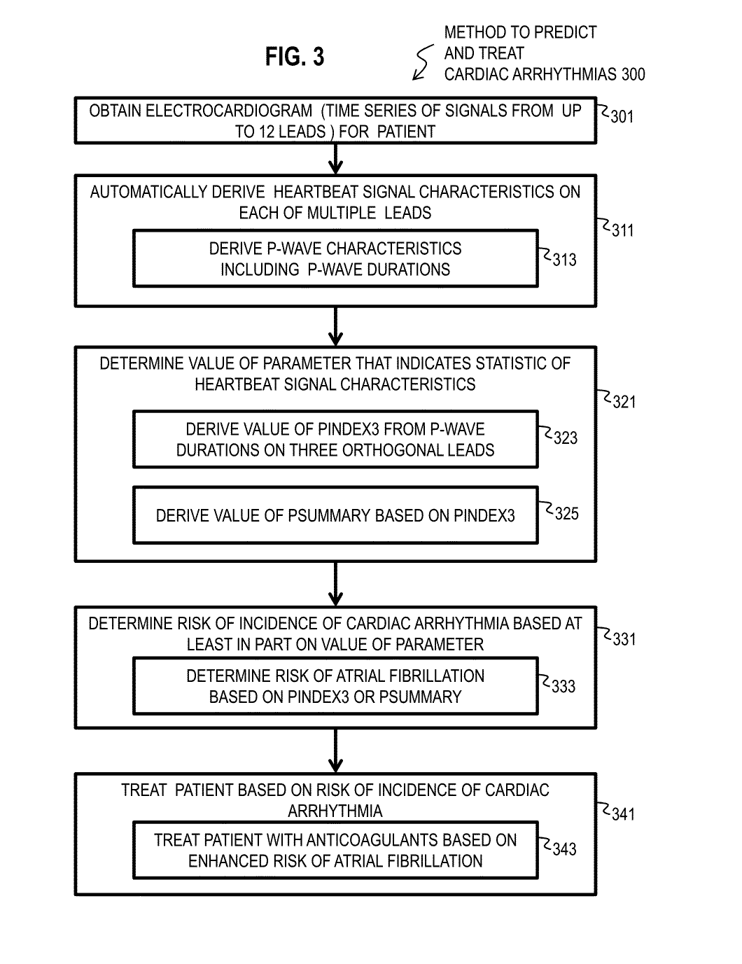

FIG. 3 is a flow diagram that illustrates an example method to predict the incidence of a cardiac arrhythmia, according to an embodiment. Although steps are depicted in FIG. 3 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways. Steps 301 through step 331 are included in arrhythmia prediction process 210, while step 341 is performed by a physician or other medical professional in response to the prediction produced by the process 210.

In step 301, an electrocardiogram is obtained for a particular patient. Any method may be used to obtain the electrocardiogram. For example, the electrocardiogram may be input manually, or scanned from an image, or communicated from an ECG controller or other device, or converted from analog data, or retrieved from a local storage device, or retrieved from a remote device or controller, in one or more messages, either unsolicited or in response to a query, or in some combination of ways. For example, one minute time series of signals on 12 leads from one patient are obtained during step 301. In some embodiments, signals from three leads only are obtained during step 301, In some of these embodiment, the signals from three orthogonal leads are obtained during step 301. Orthogonal leads are leads whose electrocardiographic vectors indicate propagation directions that are at about 90 degrees to each other in solid angle (three dimensions). A set of orthogonal leads is expected to represent most of the information in an ECG. In some embodiments, the signals from the three orthogonal leads, consisting of lead I, lead aV_{F} and lead V1, are obtained during step 301.

In step 311, heartbeat signal characteristics are automatically determined on each of the leads obtained during step 301. Algorithms are known for deriving P-wave duration of each heartbeat signal in a time series from one lead. P-wave duration is the time between the onset of the p-wave and the end of the p-wave, which corresponds to the time difference between the PR interval depicted in FIG. 1D and the PR segment depicted in FIG. 1D. Other characteristics automatically determined using known methods include P-wave axis, which relates to the direction of amplitude deflection of the P-wave in lead I and lead aV_{F} in the same heartbeat (a normal axis is a heartbeat with a positive deflection of P-wave on both leads corresponding to a direction between about zero and about 75 degrees). Another characteristic automatically derived from a lead is a premature atrial contraction, which is detected if a p-wave occurs at a time that is earlier than the next anticipated sinus p-wave. The premature atrial contraction is sometimes not followed by a set of QRS deflections. Another characteristic automatically derived from a lead using known methods is a duration for the PR interval depicted in FIG. 1D. Another characteristic automatically derived from a lead using known methods is the occurrence of a premature ventricular contraction (PVC), which is evident on a lead as a QRS complex without a preceding P-wave. Another characteristic automatically derived from a lead using known methods is the occurrence of a left bundle branch block (LBBB), which is evident as a wide QRS complex (longer than about 120 milliseconds, ms) with abnormal morphology in leads V1 and V6, such as an overall negative deflection in V1 and a positive deflection in V6.

In some embodiments, step 311 includes step 313. During step 313, P-wave durations are determined for each heartbeat in each of three orthogonal leads, such as lead I, lead aV_{F} and lead V1.

In step 321, a value is determined for a parameter that indicates a statistic of one or more heartbeat signal characteristics. In an illustrated embodiment, step 321 includes step 323. In step 323, a value is determined for a parameter Pindex3 that indicates a standard deviation of three average P-wave durations, one average duration from each of the three orthogonal leads, e.g., lead I, lead aV_{F} and lead V1. In other embodiments, Pindex3 is based on the standard deviation of individual P-wave durations on each or all of the three orthogonal leads.

In another illustrated embodiment, step 321 also includes step 325. In step 325, a value is determined for a parameter Psummary that combines multiple parameters, including Pindex3 from step 323 and age and gender, among others. In this embodiment, the other parameters include the following. The parameter abnlPaxis indicates a first value for an abnormal P-wave axis and a different second value for a normal P-wave axis, which is determined in one embodiment from just the two frontal leads I and aV_{L}. The parameter anyPAC indicates a third value in the presence of a premature atrial contraction (PAC) within a randomly chosen 10-second period among any of the ECG leads and a different fourth value in the absence of such a PAC. The parameter PRdurlong indicates elapsed time for a PR interval greater than 200 miliseconds, ms (usually, PR is determined using lead I, but selection of any other lead would be adequate since the PR is not expected to vary significantly among the different leads). The parameter Plongthree indicates a maximum P-wave duration for a maximum P-wave duration of greater than 120 ms for a longest P-wave duration among the three orthogonal leads. The parameter PVCs indicates a fifth value for a premature ventricular contraction (PVC) within a randomly chosen 10-second period among any of the 12 leads (because a PVC is expected to be evident in all leads) and a different sixth value for the absence of such a PVC. The parameter LBBB indicates a seventh value in the presence of a left bundle branch observed based on morphology of all three orthogonal leads and a different eighth value in the absence of such a left bundle branch block. In the illustrated embodiment, the first value is about 1, the second value is about 0, the third value is about 1, the fourth value is about 0, the fifth value is about 1, the sixth value is about 0, the seventh value is about 1, and the eighth value is about 0.

In one embodiment, Psummary is equal to a sum of products of an age parameter, Age, times about 0.068, and a gender parameter, Gender, times about 1.5, and Pindex3 times about 0.0056, and abnlPaxis times about 0.41, and anyPAC times about 0.74, and PRdurlong times about 0.27, and Plongthree times about 0.57 and PVCs times about 0.45, and LBBB times about 0.64. The parameter Age indicates a value for an age of the patient in years, and the parameter Gender indicates a value of about 1 if the patient is a male patient and about 0 if the patient is a female patient.

In a particular embodiment, Psummary is given by Equation 1.

Psummary=(Age parameter*0.06863)+(Gender parameter*1.48060)+(Pindex3*0.00559)+(abnlPpaxis*0.40787)+(anyPAC*0.74221)+(PRdurlong*0.26716)+(Plongthree*0.57023)+(PVCs*0.44873)+(LBBB*0.64420) (1)

In other embodiments, more or fewer parameters are included in a Psummary parameter. For example, in some embodiments one or more parameters found independently predictive, as described in more detail below, are included in a Psummary parameter.

In step 331, a risk of incidence of a cardiac arrhythmia is determined based at least in part on a value of the parameter for which a value is determined in step 321. In some embodiments, the risk is expressed as a correlation between the value of the parameter and the incidence of the arrhythmia. In some embodiments, the risk is expressed as a ratio of the odds of occurrence with and without the parameter value in a particular range. In the illustrated embodiments, the risk is expressed as a hazard ratio (HR), which offers the advantage of being a more powerful indicator of survival in the presence of a parameter value in a particular range. In survival analysis, the hazard ratio (HR) is the ratio of the hazard rates (the rate of a hazard occurring, such as the rate of atrial fibrillation occurring) corresponding to the conditions described by two levels of an explanatory variable, such as Pindex3 or Psummary. HR represents instantaneous risk over the study time period, or some subset thereof. Hazard ratios suffer somewhat less from selection bias with respect to the evaluation time (endpoint) chosen, and can indicate risks that happen before the endpoint.

For example, in some embodiments step 331 includes step 333, in which the risk is determined based on Pindex3 or Psummary, as described in more detail below. Based on statistical analysis of a historical data set, it was found that Pindex3 values greater than 35 is predictive of the incidence of atrial fibrillation in a subsequent electrocardiogram, with a HR greater than 2, compared to a Pindex3 value less than 35. Based on analysis of the same historical data set, it was found that Psummary was even more predictive. It was found that Psummary values greater than 5.4 (third quartile) is predictive of the incidence of atrial fibrillation in a subsequent electrocardiogram, with a HR greater than 5, compared to Psummary values for a first quartile. For example, the patient is at least about five times a normal risk for atrial fibrillation if the value for Psummary exceeds about 5.4. It was found that Psummary values greater than 6.3 (fourth quartile) is predictive of the incidence of atrial fibrillation in a subsequent electrocardiogram, with a HR greater than 11. For example, the patient is at least about eleven times a normal risk for atrial fibrillation if the value for Psummary exceeds about 6.3 compared to Psummary values for a first quartile.

In step 341, the patient is treated based on the risk of incidence of cardiac arrhythmia. For example, if the risk is significantly enhanced over the normal population, the patient is treated with medications that reduce the risk of the arrhythmia or reduce the harmful consequences of the arrhythmia, such as an indicated prophylaxis for AF.

In the illustrated embodiment, step 341 includes step 343 to treat the patient with anticoagulants based on enhanced risk of atrial fibrillation (e.g., Pindex3>35 or Psummary>5.4) to reduce the chances for a stroke caused by the atrial fibrillation.

The predictive power of Pindex3 and Psummary are superior to any parameters determined in the prior work. The HRs of the prior parameters using 12 leads were usually less than 2; while Pindex3 is equally predictive with fewer leads, and HR based on Psummary is greater than 5 and can exceed 11. While some of the constituent parameters of Psummary were determined to be independently predictive in earlier work, this fact merely reflects that each one has some ability to predict AF, and that even if the other factors are accounted for, it still is able to make some predictions. Though such single parameter predictions are far from complete, and it is possible that one can take two factors that have some ability to predict disease and after combining them, improve on this ability (the factors would then be considered independent and additive), it is not certain that combining them will substantially improve predictive power (e.g, improve by more than 50%). Thus, it was not anticipated that combining characteristics that were predictive with HR less than 2, or based on only three leads, such as Pindex3, would be so predictive compared to previously identified parameters. There was no indication that Pindex3 would be efficient rather than partially effective, or that the parameters of Psummary would be so cooperative and synergistic rather than redundant.

### 2. Example Embodiments

A more detailed description of the data set, material and methods isdescribed here. Some embodiments refer to 8 basic leads and 4 derived leads because one of the physical electrodes on a limb is considered a “reference” lead, used to cancel out electrical noise, but not to derive the actual ECG vectors. A more detailed description of two particular embodiments for atrial fibrillation using Pindex3 and Psummary, and their derivation, using the same data set as described in materials and methods, are also provided here.

### Material Methods and Data Sets.

Subjects without apparent AF were selected for detection of AF on a subsequent ECG. 42,751 patients found initially to be in sinus rhythm were followed for evidence of AF, with a total of 137,167 follow-up ECG recordings spanning from March 1987 to July 2000. Follow-up ECGs were also obtained for usual clinical indications. Determination of AF, both on initial ECG and on subsequent ECGs, was computerized as described below. Patients were followed until July 2000, the onset of AF, or death, whichever came first. Death was determined using the Veterans Affairs Health Care System electronic medical record or the California Health Department Service database. Study personnel blinded to ECG results determined the time of death. Review of the Veterans Affairs medical record provided a level of accuracy superior to death certificates.

There were a total of 42,751 patients with an initial ECG demonstrating absence of AF. These patients were followed for an average of 5.3 years and received a mean of 3.2 follow-up ECGs. Fifty-three percent of patients received 1 ECG, 15% had 2 ECGs, and 32% had more than 2 ECGs. The period between ECGs was, on average, 3.0 years. During this period, 1,050 (2.4%) patients were found to have AF on a subsequent ECG. Table I presents the baseline demographic information of patients who maintained sinus rhythm versus those who converted to AF. Values represent mean±SD or percentage (SE). Those who converted to AF were older (67.5 vs 55.8 years, P b 0.0001) and were less likely to be female (3.6% vs 10.5%, P b 0.0001) or Hispanic (3.9% vs 12.7%, P b 0.0046) but were more likely to be black (8.0% vs 6.2%, P b 0.0001). There were no statistically significant differences in body mass index (BMI) between the 2 groups.

TABLE 1
Baseline demographic information of patients who maintained
sinus rhythm versus those who converted to AF

| | Sinus maintained, | AF conversion, | |
|---|---|---|---|
| | n = 41,701 | n = 1,050 | P |
| Age (y) | 55.8 ± 15 | 67.55 ± 10.5 | <.0001 |
| Female | 10.5% (0.2) | 3.6% (0.6) | <.0001 |
| Height (m) | 1.74 ± 0.1 | 1.75 ± 0.09 | .0008 |
| Weight (kg) | 83.0 ± 18 | 84.2 ± 17 | .05 |
| BMI (kg/m²) | 27.3 ± 5.6 | 27.5 ± 5.5 | .35 |
| Hispanic | 12.7% (0.2) | 3.9% (0.6) | .0046 |
| Black | 6.2% (0.1) | 8.0% (0.9) | <.0001 |

P values are for differences between patients who maintained sinus rhythm and those who converted to AF. P<0.05 was considered statistically significant.

The Palo Alto Veterans Affairs Health Care System uses a computerized ECG system (GE Marquette) to collect, store, and analyze ECGs. This system has been validated by both the US Food and Drug Administration and the European Community and is widely used across the world. The current study involved the retrospective analysis of 45,855 initial ECGs obtained between March 1987 and July 2000 that were ordered for usual clinical indications. The 3,104 patients found to be in AF on the initial ECG were excluded from this analysis. Age, gender, race, weight, and height of each patient were also recorded.

The recorded data on each ECG included the timing and voltages at each of the points of the PQRST complex of the basic 8 leads with derivation of the remaining 4 leads. The system was able to flag rhythm abnormalities, measure standard intervals, and perform waveform analysis to provide the basic electrocardiographic interpretations (GE 12 SL analysis program, General Electric Company of Fairfield, Conn.). Standardized computerized ECG criteria as described by the GE 12-lead electrographic analysis program were used for the diagnosis of Q waves, ST changes, left atrial enlargement (LEA), right atrial enlargement (RAE), abnormal P axis, PAC, premature ventricular contraction (PVCs), and bundle branch blocks. From these measurements, the Romhilt-Estes criteria for left ventricular hypertrophy (LVH), spatial QRS-T angle, Cardiac Infarction Injury Scores (CIISs), Selvester scores, and the Resting ECG Neural Network (RENN) scores were calculated. Pdisp is the difference between the Pmax and Pmin, Pmean is the mean P-wave duration, and Pindex is the standard deviation (SD) of the P-wave duration across the 12 ECG leads.

### Pindex3.

Pindex3 is defined as the standard deviation of the p-wave duration from three orthogonal leads (e.g., I, aVF and V1). For the purposes of analysis of predictive power, Pindex3 values were divided into two categories: less than 35 and greater than 35. This cut-off was chosen given the predictive power seen in the former parameter Pindex that is based on the standard deviation of p-wave duration among all 12 leads of the 12-lead electrocardiogram.

As shown in Table 2, on multivariate analysis, after adjusting for all other factors (including Age and Gender) which were shown to be predictive of AF in the 12-lead ECG analysis, Pindex3>35 is still independently predictive of incident atrial fibrillation with a Hazard Ratio of 1.34. This establishes the independent predictive power of this novel variable. AbnlPaxis is defined as a p-wave axis less than 0 degrees or greater than 75 degrees. AnyPAC is any premature atrial contraction present on the 12-lead ECG (yes/no), PRdurlong is defined as a PR interval greater than 200 ms (yes/no), PVCs is any premature ventricular contraction present (yes/no), and LBBB is left bundle branch block (yes/no).

TABLE 2
Predictive power of individual parameters in multivariate analysis

| | | Parameter | Standard | Chi- | | Hazard | 95% Hazard Ratio | |
|---|---|---|---|---|---|---|---|---|
| Parameter | DF | Estimate | Error | Square | Pr > ChiSq | Ratio | Confidence Limits | |
| Age | 1 | 0.06917 | 0.00284 | 594.0224 | <.0001 | 1.072 | 1.066 | 1.078 |
| Gender | 1 | 1.51647 | 0.18920 | 64.2417 | <.0001 | 4.556 | 3.144 | 6.601 |
| Pindex3 > 35 | 1 | 0.29213 | 0.06635 | 19.3857 | <.0001 | 1.339 | 1.176 | 1.525 |
| abnlPaxis | 1 | 0.68244 | 0.11224 | 36.9704 | <.0001 | 1.979 | 1.588 | 2.466 |
| AnyPAC | 1 | 0.75628 | 0.12678 | 35.5870 | <.0001 | 2.130 | 1.662 | 2.731 |
| prdurlong | 1 | 0.44331 | 0.09325 | 22.6033 | <.0001 | 1.558 | 1.298 | 1.870 |
| PVCs | 1 | 0.50912 | 0.11188 | 20.7065 | <.0001 | 1.664 | 1.336 | 2.072 |
| LBBB | 1 | 0.69784 | 0.17653 | 15.6270 | <.0001 | 2.009 | 1.422 | 2.840 |

### Psummary.

In this embodiment, Psummary is the value calculated using Equation 1. Where Age is expressed in years, Gender carries a value of 1 for males and 0 for females, Pindex3 is the standard deviation of the p-wave duration from three orthogonal leads (e.g., I, aVF and V1), abnlPaxis carries the value of 1 for an abnormal p-wave axis and 0 for a normal p-wave axis, AnyPAC carries a value of 1 in the presence of a premature atrial contraction within a randomly chosen 10-second period and a value of 0 in the absence of such a premature atrial contraction, PRdurlong is a PR interval greater than 200 miliseconds, Plongthree is a maximum p-wave duration of greater than 120 ms, PVCs carries a value of 1 for a premature ventricular complex within a randomly chosen 10-second period and a value of 0 for the absence of such a ventricular premature complex, and LBBB carries a value of 1 in the presence of a left bundle branch block and 0 in the absence of a left bundle branch block.

This embodiment assumes that the measurements were made using an orthogonal 3-lead system (e.g., lead I, aVF and V1). In other embodiments, similar formulas are derived from other orthogonal 3-lead sets, or a 12-lead ECG. Also, in some embodiments, similar formulas are derived with variation in the terms used, by subtracting variables included, or by substituting or adding similar variables that factor amplitude and duration of the p-wave or p-wave/QRS relationship.

The parameters for this embodiment were estimated from a multivariate Cox regression model predicting incident atrial fibrillation as listed in Table 3. In other embodiments, values of the parameters are anticipated to vary up or down based on the Standard Error as noted in Table 3.

TABLE 3
Analysis of Maximum Likelihood Estimates for individual parameters

| | | Parameter | Standard | Chi- | | Hazard | 95% Hazard Ratio | |
|---|---|---|---|---|---|---|---|---|
| Parameter | DF | Estimate | Error | Square | Pr > ChiSq | Ratio | Confidence Limits | |
| Age | 1 | 0.06863 | 0.00292 | 551.1968 | <.0001 | 1.071 | 1.065 | 1.077 |
| Gender | 1 | 1.48060 | 0.19268 | 59.0501 | <.0001 | 4.396 | 3.013 | 6.412 |
| psdthree | 1 | 0.00559 | 0.00275 | 4.1418 | 0.0418 | 1.006 | 1.000 | 1.011 |
| abnlpaxis | 1 | 0.40787 | 0.13956 | 8.5411 | 0.0035 | 1.504 | 1.144 | 1.977 |
| AnyPAC | 1 | 0.74221 | 0.13652 | 29.5576 | <.0001 | 2.101 | 1.607 | 2.745 |
| PRdurlong | 1 | 0.26716 | 0.10106 | 6.9882 | 0.0082 | 1.306 | 1.072 | 1.592 |
| Plongthree | 1 | 0.57023 | 0.08263 | 47.6252 | <.0001 | 1.769 | 1.504 | 2.080 |
| PVCs | 1 | 0.44873 | 0.11763 | 14.5527 | 0.0001 | 1.566 | 1.244 | 1.972 |
| LBBB | 1 | 0.64420 | 0.18758 | 11.7945 | 0.0006 | 1.904 | 1.319 | 2.751 |

The predictive power of Psummary was calculated by dividing Psummary into quartiles and running a Cox Hazard Regression model with quartiles 2-4 compared to the first quartile. This was done to create cutoffs in an un-biased manner. The predictive power of the quartiles is shown in Table 4, after adjusting for Age and Gender. Of important note, the results that were observed were well beyond what would have been expected for any single factor, and the great degree of predictive power was surprising. For the third quartile (Psummary between about 5.4 and about 6.3), the Hazard Ratio was 5.3, and for the fourth quartile (Psummary>6.3), the Hazard Ratio was 11.1. This means that, after adjusting for age and sex, a person with a Psummary that falls in the fourth quartile (Psummary>6.3) would be expected to have an 11-fold greater chance of developing atrial fibrillation compared to somebody in the first quartile.

TABLE 4
Analysis of Maximum Likelihood Estimates for Psummary quartiles

| | | Parameter | Standard | Chi- | | Hazard | 95% Hazard Ratio | |
|---|---|---|---|---|---|---|---|---|
| Parameter | DF | Estimate | Error | Square | Pr > ChiSq | Ratio | Confidence Limits | |
| Age | 1 | 0.02820 | 0.00556 | 25.7208 | <.0001 | 1.029 | 1.017 | 1.040 |
| Gender | 1 | 0.45448 | 0.21864 | 4.3208 | 0.0376 | 1.575 | 1.026 | 2.418 |
| PsummaryQuartile 2 | 1 | 0.68058 | 0.24175 | 7.9258 | 0.0049 | 1.975 | 1.230 | 3.172 |
| PsummaryQuartile 3 | 1 | 1.68152 | 0.24531 | 46.9860 | <.0001 | 5.374 | 3.323 | 8.691 |
| PsummaryQuartile 4 | 1 | 2.41010 | 0.28022 | 73.9751 | <.0001 | 11.135 | 6.429 | 19.285 |

### 3. Hardware Overview

FIG. 4 is a block diagram that illustrates a computer system 400 upon which an embodiment of the invention may be implemented. Computer system 400 includes a communication mechanism such as a bus 410 for passing information between other internal and external components of the computer system 400. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 400, or a portion thereof, constitutes a means for performing one or more steps of one or more methods described herein.

A sequence of binary digits constitutes digital data that is used to represent a number or code for a character. A bus 410 includes many parallel conductors of information so that information is transferred quickly among devices coupled to the bus 410. One or more processors 402 for processing information are coupled with the bus 410. A processor 402 performs a set of operations on information. The set of operations include bringing information in from the bus 410 and placing information on the bus 410. The set of operations also typically include comparing two or more units of information, shifting positions of units of information, and combining two or more units of information, such as by addition or multiplication. A sequence of operations to be executed by the processor 402 constitutes computer instructions.

Computer system 400 also includes a memory 404 coupled to bus 410. The memory 404, such as a random access memory (RAM) or other dynamic storage device, stores information including computer instructions. Dynamic memory allows information stored therein to be changed by the computer system 400. RAM allows a unit of information stored at a location called a memory address to be stored and retrieved independently of information at neighboring addresses. The memory 404 is also used by the processor 402 to store temporary values during execution of computer instructions. The computer system 400 also includes a read only memory (ROM) 406 or other static storage device coupled to the bus 410 for storing static information, including instructions, that is not changed by the computer system 400. Also coupled to bus 410 is a non-volatile (persistent) storage device 408, such as a magnetic disk or optical disk, for storing information, including instructions, that persists even when the computer system 400 is turned off or otherwise loses power.

Information, including instructions, is provided to the bus 410 for use by the processor from an external input device 412, such as a keyboard containing alphanumeric keys operated by a human user, or a sensor. A sensor detects conditions in its vicinity and transforms those detections into signals compatible with the signals used to represent information in computer system 400. Other external devices coupled to bus 410, used primarily for interacting with humans, include a display device 414, such as a cathode ray tube (CRT) or a liquid crystal display (LCD), for presenting images, and a pointing device 416, such as a mouse or a trackball or cursor direction keys, for controlling a position of a small cursor image presented on the display 414 and issuing commands associated with graphical elements presented on the display 414.

In the illustrated embodiment, special purpose hardware, such as an application specific integrated circuit (IC) 420, is coupled to bus 410. The special purpose hardware is configured to perform operations not performed by processor 402 quickly enough for special purposes. Examples of application specific ICs include graphics accelerator cards for generating images for display 414, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 400 also includes one or more instances of a communications interface 470 coupled to bus 410. Communication interface 470 provides a two-way communication coupling to a variety of external devices that operate with their own processors, such as printers, scanners and external disks. In general the coupling is with a network link 478 that is connected to a local network 480 to which a variety of external devices with their own processors are connected. For example, communication interface 470 may be a parallel port or a serial port or a universal serial bus (USB) port on a personal computer. In some embodiments, communications interface 470 is an integrated services digital network (ISDN) card or a digital subscriber line (DSL) card or a telephone modem that provides an information communication connection to a corresponding type of telephone line. In some embodiments, a communication interface 470 is a cable modem that converts signals on bus 410 into signals for a communication connection over a coaxial cable or into optical signals for a communication connection over a fiber optic cable. As another example, communications interface 470 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 470 sends and receives electrical, acoustic or electromagnetic signals, including infrared and optical signals, that carry information streams, such as digital data.

The term computer-readable medium is used herein to refer to any medium that participates in providing information to processor 402, including instructions for execution. Such a medium may take many forms, including, but not limited to, non-volatile media, volatile media and transmission media. Non-volatile media include, for example, optical or magnetic disks, such as storage device 408. Volatile media include, for example, dynamic memory 404. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 402, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 402, except for carrier waves and other signals.

Logic encoded in one or more tangible media includes one or both of processor instructions on a computer-readable storage media and special purpose hardware, such as ASIC 420.

Network link 478 typically provides information communication through one or more networks to other devices that use or process the information. For example, network link 478 may provide a connection through local network 480 to a host computer 482 or to equipment 484 operated by an Internet Service Provider (ISP). ISP equipment 484 in turn provides data communication services through the public, world-wide packet-switching communication network of networks now commonly referred to as the Internet 490. A computer called a server 492 connected to the Internet provides a service in response to information received over the Internet. For example, server 492 provides information representing video data for presentation at display 414.

The invention is related to the use of computer system 400 for implementing the techniques described herein. According to one embodiment of the invention, those techniques are performed by computer system 400 in response to processor 402 executing one or more sequences of one or more instructions contained in memory 404. Such instructions, also called software and program code, may be read into memory 404 from another computer-readable medium such as storage device 408. Execution of the sequences of instructions contained in memory 404 causes processor 402 to perform the method steps described herein. In alternative embodiments, hardware, such as application specific integrated circuit 420, may be used in place of or in combination with software to implement the invention. Thus, embodiments of the invention are not limited to any specific combination of hardware and software.

The signals transmitted over network link 478 and other networks through communications interface 470, carry information to and from computer system 400. Computer system 400 can send and receive information, including program code, through the networks 480, 490 among others, through network link 478 and communications interface 470. In an example using the Internet 490, a server 492 transmits program code for a particular application, requested by a message sent from computer 400, through Internet 490, ISP equipment 484, local network 480 and communications interface 470. The received code may be executed by processor 402 as it is received, or may be stored in storage device 408 or other non-volatile storage for later execution, or both. In this manner, computer system 400 may obtain application program code in the form of a signal on a carrier wave.

Various forms of computer readable media may be involved in carrying one or more sequence of instructions or data or both to processor 402 for execution. For example, instructions and data may initially be carried on a magnetic disk of a remote computer such as host 482. The remote computer loads the instructions and data into its dynamic memory and sends the instructions and data over a telephone line using a modem. A modem local to the computer system 400 receives the instructions and data on a telephone line and uses an infra-red transmitter to convert the instructions and data to a signal on an infra-red a carrier wave serving as the network link 478. An infrared detector serving as communications interface 470 receives the instructions and data carried in the infrared signal and places information representing the instructions and data onto bus 410. Bus 410 carries the information to memory 404 from which processor 402 retrieves and executes the instructions using some of the data sent with the instructions. The instructions and data received in memory 404 may optionally be stored on storage device 408, either before or after execution by the processor 402.

FIG. 5 illustrates a chip set 500 upon which an embodiment of the invention may be implemented. Chip set 500 is programmed to perform one or more steps of a method described herein and includes, for instance, the processor and memory components described with respect to FIG. 4 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 500, or a portion thereof, constitutes a means for performing one or more steps of a method described herein.

In one embodiment, the chip set 500 includes a communication mechanism such as a bus 501 for passing information among the components of the chip set 500. A processor 503 has connectivity to the bus 501 to execute instructions and process information stored in, for example, a memory 505. The processor 503 may include one or more processing cores with each core configured to perform independently. A multi-core processor enables multiprocessing within a single physical package. Examples of a multi-core processor include two, four, eight, or greater numbers of processing cores. Alternatively or in addition, the processor 503 may include one or more microprocessors configured in tandem via the bus 501 to enable independent execution of instructions, pipelining, and multithreading. The processor 503 may also be accompanied with one or more specialized components to perform certain processing functions and tasks such as one or more digital signal processors (DSP) 507, or one or more application-specific integrated circuits (ASIC) 509. A DSP 507 typically is configured to process real-world signals (e.g., sound) in real time independently of the processor 503. Similarly, an ASIC 509 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 503 and accompanying components have connectivity to the memory 505 via the bus 501. The memory 505 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 505 also stores the data associated with or generated by the execution of one or more steps of the methods described herein.

### 4. Extensions, Modifications and Alternatives.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

1. A method comprising:
obtaining first data that indicates an electrocardiography recording from a patient;
automatically deriving, on a processor, P-wave characteristics on a plurality of leads of the electrocardiography recording;
determining a value for a first parameter, called Pindex3, based on a standard deviation of P-wave durations automatically derived from only three leads of the plurality of leads;
determining a risk of incidence of cardiac arrhythmia for the patient based, at least in part, on the first parameter, Pindex3; and
presenting on a display device output data that indicates the risk of incidence.

2. A method as recited in claim 1, wherein the only three leads are orthogonal to each other.

3. A method as recited in claim 1, wherein the only three leads consist of lead I, lead aVF and lead V1 of the electrocardiography recording from the patient.

4. A method as recited in claim 1, further comprising treating the patient based on the determined risk of incidence of cardiac arrhythmia.

5. A method as recited in claim 4, wherein:
determining the risk of incidence of cardiac arrhythmia for the patient further comprises determining that the patient is at elevated risk for atrial fibrillation if the value for the first parameter exceeds 45; and
treating the patient further comprises treating the patient to reduce risk of atrial fibrillation if the patient is determined to be at elevated risk for atrial fibrillation.

6. A method as recited in claim 1, wherein:
the method further comprises determining a value for a different second parameter based on P-wave characteristics automatically derived from at least one of the plurality of leads; and
determining the risk of incidence of cardiac arrhythmia for the patient further comprises determining the risk of incidence of cardiac arrhythmia for the patient based at least in part on the second parameter.

7. A method as recited in claim 6, wherein the different second parameter is selected from a group comprising:
abnlPaxis that indicates a first value for an abnormal P-wave axis and a different second value for a normal P-wave axis;
anyPAC that indicates a third value in the presence of a premature atrial contraction (PAC) within a randomly chosen 10-second period and a different fourth value in the absence of the premature atrial contraction (PAC);
PRdurlong that indicate a PR interval greater than 200 miliseconds;
Plongthree that indicates a maximum P-wave duration of greater than 120 ms;
PVCs that indicates a fifth value for a premature ventricular contraction within a randomly chosen 10-second period and a different sixth value for the absence of such a ventricular premature complex; and
LBBB that indicates a seventh value in the presence of a left bundle branch block and a different eighth value in the absence of a left bundle branch block.

8. A method as recited in claim 7, wherein:
the first value is about 1, the second value is about 0, the third value is about 1, the fourth value is about 0, the fifth value is about 1, the sixth value is about 0, the seventh value is aboutl, and the eighth value is about 0;
determining the risk of incidence of cardiac arrhythmia for the patient further comprises determining the risk of incidence of atrial fibrillation for the patient based on a parameter Psummary equal to a sum of products of an age parameter times about 0.068, and a gender parameter times about 1.5, and Pindex3 times about 0.0056, and abnlPaxis times about 0.41, and anyPAC times about 0.74, and PRdurlong times about 0.27, and Plongthree times about 0.57 and PVCs times about 0.45, and LBBB times about 0.64; and
the age parameter indicates a value for an age of the patient in years, and the gender parameter indicates a value of about 1 if the patient is a male patient and about 0 if the patient is a female patient.

9. A method as recited in claim 8, wherein Psummary=(Age parameter*0.06863)+(Gender parameter*1.48060)+(Pindex3*0.00559)+(abnlPpaxis*0.40787)+(anyPAC*0.74221)+(PRdurlong*0.26716)+(Plongthree*0.57023)+(PVCs*0.44873)+(LBBB*0.64420).

10. A method as recited in claim 7, wherein determining the risk of incidence of atrial fibrillation for the patient based on the parameter Psummary further comprises determining that the patient is at least about five times a normal risk for atrial fibrillation if the value for Psummary exceeds about 5.4.

11. A method as recited in claim 7, wherein determining the risk of incidence of atrial fibrillation for the patient based on the parameter Psummary further comprises determining that the patient is at least about eleven times a normal risk for atrial fibrillation if the value for Psummary exceeds about 6.3.

12. A method as recited in claim 1, wherein the cardiac arrhythmia is atrial fibrillation.

13. A method as recited in claim 1, wherein the cardiac arrhythmia is atrial flutter.

14. A non-transitory computer-readable medium carrying one or more sequences of instructions, wherein execution of the one or more sequences of instructions by one or more processors causes an apparatus to perform the steps of:
obtaining first data that indicates an electrocardiography recording from a patient;
automatically deriving, on a processor, P-wave characteristics on a plurality of leads of the electrocardiography recording;
determining a value for a first parameter, Pindex3, based on a standard deviation of P-wave durations automatically derived from only three leads of the plurality of leads;
determining a risk of incidence of cardiac arrhythmia for the patient based, at least in part, on the first parameter; and
presenting on a display device output data that indicates the risk of incidence.

15. An apparatus comprising:
at least one processor; and
at least one memory including one or more sequences of instructions,
the at least one memory and the one or more sequences of instructions configured to, with the at least one processor, cause the apparatus to perform at least the following, obtaining first data that indicates an electrocardiography recording from a patient;
automatically deriving, on a processor, P-wave characteristics on a plurality of leads of the electrocardiography recording;
determining a value for a first parameter, Pindex3, based on a standard deviation of P-wave durations automatically derived from only three leads of the plurality of leads;
determining a risk of incidence of cardiac arrhythmia for the patient based, at least in part, on the first parameter; and
presenting on a display device output data that indicates the risk of incidence.

16. A system comprising:
means for obtaining first data that indicates an electrocardiography recording from a patient;
means for automatically deriving, on a processor, P-wave characteristics on a plurality of leads of the electrocardiography recording;
means for determining a value for a first parameter, Pindex3, based on a standard deviation of P-wave durations automatically derived from only three leads of the plurality of leads;
means for determining a risk of incidence of cardiac arrhythmia for the patient based, at least in part, on the first parameter; and
means for presenting on a display device output data that indicates the risk of incidence.

17. A system comprising:
a display device;
at least one processor; and
at least one memory including one or more sequences of instructions,
the at least one memory and the one or more sequences of instructions configured to, with the at least one processor, cause the apparatus to perform at least the following, obtaining first data that indicates an electrocardiography recording from a patient;
automatically deriving, on a processor, P-wave characteristics on a plurality of leads of the electrocardiography recording;
determining a value for a first parameter, Pindex3, based on a standard deviation of P-wave durations automatically derived from only three leads of the plurality of leads;
determining a risk of incidence of cardiac arrhythmia for the patient based, at least in part, on the first parameter; and
presenting on the display device output data that indicates the risk of incidence.
